(19) United States
(12) Patent Application Publication
(10) Pub. No.: US 2019/0313936 A1
(43) Pub. Date: Oct. 17, 2019

(54) SIMPLE METHOD TO MEASURE MAGNETIC RESONANCE ANISOTROPIC T2 RELAXATION TIMES IN HUMAN KNEE CARTILAGE

(71) Applicant: THE REGENTS OF THE UNIVERSITY OF MICHIGAN, Ann Arbor, MI (US)

(72) Inventors: Yuxi Pang, Superior Township, MI (US); Thomas L. Chenevert, Ann Arbor, MI (US); Riann Palmieri-Smith, Ann Arbor, MI (US)

(21) Appl. No.: 16/385,981

(22) Filed: Apr. 16, 2019

Related U.S. Application Data
(60) Provisional application No. 62/658,210, filed on Apr. 16, 2018.

(51) Int. Cl. A61B 5/055; A61B 5/00; G01R 33/50; G01R 33/44

(52) U.S. Cl. CPC A61B 5/055; A61B 5/4585; G01R 33/50; G01R 33/56358; G01R 33/445; A61B 5/7282; G01R 33/448

(57) ABSTRACT

Technology for measuring clinically relevant Magnetic Resonance (MR) anisotropic water proton transverse relaxation rates of cartilage in human knees and other joints is provided. A single sagittal T2-weighted magnetic resonance image of a cartilage associated with a human knee or other joint may be acquired, and an internal reference signal may be measured from this acquired sagittal image. The internal reference signal is a signal intensity (S(θ))from the cartilage with its collagen fiber orientated approximately 54.7° to the main magnetic field. An anisotropic R₂ relaxation rate (R(θ)₂^{a}) may be calculated for the cartilage using the following equation: R(θ)₂^{a}={log(S(θ=±54.7°))−log(S(θ))}/TE. Accordingly, an indication of a degeneration of the cartilage may be determined using the calculated anisotropic R2 relaxation rate.

### CROSS-REFERENCE TO RELATED APPLICATIONS

This application claims the benefit of provisional U.S. Patent Application No. 62/658,210, which is entitled “A SIMPLE METHOD TO MEASURE MAGNETIC RESONANCE ANISOTROPIC T2 RELAXATION TIMES IN HUMAN KNEE CARTILAGE,” which was filed on Apr. 16, 2018, and the disclosure of which is hereby expressly incorporated by reference herein in its entirety.

### FIELD OF THE DISCLOSURE

The present disclosure generally relates to a method of measuring clinically relevant Magnetic Resonance (MR) anisotropic water proton transverse relaxation rates of cartilage in human knee and other joints.

### BACKGROUND

Osteoarthritis (OA) is a joint disease with a hallmark of progressive loss of articular cartilage—a highly specialized connective tissue whose primary function is to facilitate smooth and effortless joint motions. Because cartilage is avascular and does not contain any nerves, early cartilage degeneration may be asymptomatic and a potential repair process would be slow following tissue damage. No cure exists for OA to date; therefore, it would be extremely beneficial to have a noninvasive imaging method to detect pre-structural cartilage changes as early as possible, so that a timely effective intervention could be available to patients to slow down adverse progression in cartilage. Additionally, a noninvasive imaging method could also be used as a surrogate biomarker to monitor both therapeutic outcomes and possible progression to OA after anterior cruciate ligament (ACL) reconstruction surgery.

Considerable multidisciplinary efforts have been devoted in recent years to cartilage characterizations using high-field nuclear magnetic resonance (NMR) and magnetic resonance (MR) imaging techniques. Particularly, quantitative MR imaging techniques are attractive due to their unique capacities to access compositional changes in cartilage, which is critical in detecting early cartilage degeneration at a molecular level. To this point, a series of developed MR methods have leveraged on specific physical properties of cartilage, for instances, both sodium MR imaging and delayed gadolinium-enhanced MRI of cartilage (dGEMRIC) depend on an amount of negatively charged molecules on proteoglycan (mostly glycosaminoglycans, GAGs) in extracellular matrix (ECM). These two MR techniques have either an intrinsic relatively low signal sensitivity or a lengthy acquisition protocol besides a required exogenous contrast agent administration. The chemical exchange effect between labile hydroxyl (—OH) group and bulk water in cartilage has been exploited in chemical exchange saturation transfer imaging of GAG (gagCEST), which was reported having less than 10% effect at 7T and was negligible at 3T. Another high-field advanced MR application is quantitative susceptibility mapping (QSM), which heavily relies on an intrinsic magnetic susceptibility anisotropy within the helical polypeptides in cartilage fibrils. However, this promising MR technique could be challenging to use in routine clinical applications due to a decreased effect at 3T and a demanding image post-processing workflow.

Water proton T1 and T2 relaxation times in pure liquids are primarily determined by modulation of the intramolecular dipole-dipole interaction created by molecular isotropic re-orientation and characterized by a rotational correlation time τ_{c}. In biological tissue, magnetization transfer between water and immobilized components affects T₁ relaxation times, and water exchange between free and restricted domains alters T₂ relaxation times. In addition, intricate cellular and micro-structural arrangements can restrict molecular reorientation of water, creating an orientation-dependent T₂ and T_{1ρ} (spin-lattice relaxation time in a rotating frame) in highly organized tissues such as skeletal muscles and collagen fibers. An efficient method for quick quantification of MR anisotropic relaxation could provide invaluable insights into the integrity of structured tissues.

Articular cartilage primarily comprises water (˜68-85% total weight), structural proteins including mostly collagen (60-80% dry weight) and proteoglycans (˜15-40% dry weight), and a sparse distribution (˜2%) of chondrocytes. Proteoglycans consist of a core with one or more negatively charged linear glycosaminoglycan (GAG) chains covalently attached. In contrast, collagen forms fibrils and fibers intertwined with proteoglycans. Histologically, articular cartilage could be divided into the superficial (SZ), transitional (TZ), and deep (DZ) zones, where collagen fibers are, respectively, orientated in parallel, randomly, and perpendicularly with respect to the cartilage surface. These highly organized collagen fibers, particularly in the DZ, create an anisotropic environment for the vast amount of water in cartilage, resulting in reported MR relaxation anisotropies.

The orientation-dependent MR relaxation rates R₁ (1/T1), R₂ (1/T2), and R_{1ρ} (1/T2) in bovine patellar cartilage-bone specimens have been recently characterized at 9.4T. R_{1ρ} was determined with both constant amplitude and adiabatic waveforms as a function of spin-lock RF power. This study shows that the relaxation rates R₁ and R₂ had minimal and maximal orientation dependences, respectively. The orientation anisotropy of R_{1ρ} was almost completely suppressed if a stronger spin-lock RF field was used. More importantly, the relaxation parameters with higher orientation anisotropies were found to be more sensitive to cartilage degenerative changes secondary to osteoarthritis (OA). In other words, the anisotropic component of R₂ (i.e., R^{a}₂ (θ)) has the potential to be a more sensitive MRI biomarker for early cartilage changes in OA and a valuable imaging tool to follow OA progression after anterior cruciate ligament (ACL) reconstruction surgery.

In conventional R₂ mapping, R^{a}₂ (θ) is not separated from its isotropic counterpart, potentially compromising the sensitivity and specificity of the measure. R₂ and R_{1ρ} are currently the most investigated relaxation metrics in clinical studies of knee cartilage degeneration, but the interpretation of R₂ and R_{1ρ} measurements in terms of observed structural protein changes is not straightforward. Most likely, neither R₂ nor R_{1ρ} has sufficient sensitivity to the underlying biochemical and physiological changes in cartilage. To increase the sensitivity of MR detection of OA, a composite relaxation metric, R₂−R_{1ρ}, has been proposed. Subtracting R_{1ρ} from R₂ removes the isotropic contribution to R₂ to a certain extent if the spin-lock RF used in R_{1ρ} mapping is not strong enough. Previous work has proposed that R_{1ρ} is driven by exchange of hydroxyl protons in GAG with bulk water protons. This hypothesis seems consistent with exchange-driven mechanisms that determine R_{1ρ} but conflicts with previous findings in cartilage.

Chemical shift increases linearly with increasing magnetic field (B₀), and the relaxation rate R₂, due to exchange between protons with different chemical shifts, increases quadratically with B₀. In contrast, the contribution from dipolar interaction to R₂ is mostly independent of B₀. Provided that an increased R₂ at a higher B₀ could be attributed entirely to the chemical exchange effect, a comparison of R₂ at two different B₀ should shed light on the relative importance of two different relaxation mechanisms. Mlynarik et al. performed a detailed study on R₂ and R_{1ρ} of human cartilage-bone specimens at 2.95T and 7.05T and concluded that the (residual) dipolar interaction was the dominant relaxation mechanism at B₀≤3T.

Later, two clinical studies on healthy human knee cartilage showed that R₂ at 7T was either close to or 18% larger than that at 3T, suggesting that the chemical exchange effect would have contributed less than 4% to R₂ if it had been measured at 3T. A similar finding was reported for R_{1ρ} of healthy human knee cartilage, with less than 15% increase at 7T relative to 3T. Furthermore, the chemical exchange effect on R₂ at 3T can be simulated using published parameters, i.e., H2O of 88 [M], GAG of 0.3 [M], exchange rate of 1 kHz, and chemical shift of 1 ppm, and it turned out to be a negligible value of 0.05 (1/s) compared with the observed R₂ of about 30 (1/s).

### SUMMARY

A computer-implemented method is provided. The computer-implemented method includes acquiring a single sagittal T2-weighted magnetic resonance image of a human knee cartilage, and measuring an internal reference signal taken from the deep zone in femoral cartilage with the cartilage fiber orientated approximately 54.7° to the external main magnetic field. The anisotropic R₂ relaxation rate (R(θ)₂^{a}) for the human knee femoral cartilage could be obtained using the following equation: R(θ)₂^{a}={log(S(θ=±54.7°))−log(S(θ))}/TE, which indicates an integrity of—human knee femoral cartilage. The R(θ)₂^{a} for other knee cartilage could be measured using the same determined internal reference. The proposed method is also applied to other joints cartilage.

In one aspect, a computer-implemented method is provided. The computer-implemented method comprises: acquiring a single sagittal T2-weighted magnetic resonance image of cartilage associated with a human joint; measuring an internal reference signal in this acquired sagittal image, wherein the internal reference signal is a signal intensity (S(θ)) from the cartilage with its collagen fiber orientated approximately 54.7° to the main magnetic field; calculating an anisotropic R₂ relaxation rate (R(θ)₂^{a}) for the cartilage, using the following equation: R(θ)₂^{a}={log(S(θ=±54.7°))−log(S(θ))}/TE; and determining an indication of a degeneration of the cartilage using the calculated anisotropic R₂ relaxation rates.

In another aspect, a system is provided. The system comprises one or more processors and one or more memories. The memories store instructions that, when executed by the one or more processors, cause the one or more processors to acquire a single sagittal T2-weighted magnetic resonance image of cartilage associated with a human joint; measure an internal reference signal in this acquired sagittal image, wherein the internal reference signal is a signal intensity (S(θ)) from the cartilage with its collagen fiber orientated approximately 54.7° to the main magnetic field; calculate an anisotropic R₂ relaxation rate (R(θ)₂^{a}) for the cartilage, using the following equation: R(θ)₂^{a}={log(S(θ=±54.7°))−log(S(θ))}/TE; and determine an indication of a degeneration of the human knee cartilage using the calculated anisotropic R₂ relaxation rates.

In still another aspect, a tangible, non-transitory computer-readable medium storing executable instructions is provided. The instructions, when executed by at least one processor of a computing device, cause the computing device to: acquire a single sagittal T2-weighted magnetic resonance image of cartilage associated with a human joint; measure an internal reference signal in this acquired sagittal image, wherein the internal reference signal is a signal intensity (S(θ)) from the cartilage with its collagen fiber orientated approximately 54.7° to the main magnetic field; calculate an anisotropic R₂ relaxation rate (R(θ)₂^{a}) for the cartilage, using the following equation: R(θ)₂^{a}={log(S(θ=±54.7°))−log(S(θ))}/TE; and determine an indication of a degeneration of the cartilage using the calculated anisotropic R₂ relaxation rates.

### BRIEF DESCRIPTION OF THE DRAWINGS

FIG. 1 illustrates an exemplary schematic diagram of femoral articular cartilage with angular-radial segmentations, anatomical annotations and collagen fiber orientations. The deep and superficial zones are divided by a red dash-dot line. An ROI, at a magic-angle of −54.7° with an angular width of 5° in the deep zone, is represented by a red square. The main magnetic field B₀ points downwards.

FIG. 2 illustrates an exemplary segmented T2W signal (in a logarithmic scale) intensity map for a whole deep cartilage from the one knee (b). A white arrow points to the spatial location of an angular-radial segmentations in a lateral T2W image (a), and two horizontal dashed lines cross the segmented angles φ of ±54.7°.

FIG. 3 illustrates exemplary (a) R₂, (b) R_{1ρ}, (c) R₂−R_{1ρ} and (d) R(θ)₂^{a} pixel maps for the image in FIG. 2a. An internal reference of 5.52 and TE of 48.8 ms was used in derivingR(θ)₂^{a}. All color bars are in the same scale.

FIG. 4 illustrates exemplary comparisons between orientation-dependent relaxation rate profiles of R₂ (blue circles) and R_{1ρ} (red squares) in the deep (a) and superficial (b) zones, and the similar comparisons between R(θ)₂^{a} (red squares) and R₂−R_{1ρ} (blue circles) in the deep (c) and the superficial (d) zones. Each point in the plots was an averaged relaxation rate from a segmented ROI in the corresponding relaxation rate maps shown in FIG. 3.

FIG. 5 illustrates exemplary whole relaxation rate (s⁻¹) maps for (a, e) R₂, (b, f) R_{1ρ}, (c, g) R₂−R_{1ρ} and (d, h) R(θ)₂^{a} in the deep (a-d) and superficial (e-h) cartilage from one subject. All color bars are in the same scale.

FIG. 6 illustrates exemplary correlations between R₂−R_{1ρ} from FIG. 5c,g and R(θ)₂^{a} from FIG. 5d,h in the deep (a) and superficial (b) cartilage, with the linear Pearson correlation coefficients (r) included. Data ellipses are shown in red dashed lines, with the means of R₂−R_{1ρ} and R(θ)₂^{a} highlighted by the horizontal and the vertical green dashed lines.

FIG. 7 illustrates exemplary comparisons of the whole-knee averaged relaxation rates (s⁻¹) between R(θ)₂^{a} (circle and square) and R₂−R_{1ρ} (triangle and diamond) in the deep (solid line) and superficial (dashed lines) cartilage for five knees.

FIG. 8 illustrates exemplary comparisons of the normalized whole-knee averaged relaxation rates (%) between R(θ)₂^{a} (yellow) and R₂−R_{1ρ} (blue) for examined five knees. The normalized quantity (%) was defined as (DEEP-SUPF)/DEEP*100, with DEEP and SUPF representing relaxation rates in the deep and superficial cartilage.

FIG. 9 illustrates an exemplary computer system that may be used for analysis as described here and connected to a medical imaging system.

FIG. 10 illustrates a flow diagram of an exemplary method for measuring clinically relevant Magnetic Resonance (MR) anisotropic water proton transverse relaxation rates of cartilage in human knee and other joints.

### DETAILED DESCRIPTION

The present disclosure provides an efficient method to measure the collagen orientation-dependent anisotropic transverse water proton relaxation rates in human knee cartilage. Advantageously, this method may reduce clinical MR scan times significantly and may allow clinicians and researchers to derive more relevant and sensitive information on collagen integrity of both knees and other joints.

The present disclosure first demonstrates theoretically that the R^{a}₂ (θ) of cartilage at 3T was partially and inefficiently separated in the reported composite relaxation metric. The prolonged image acquisition protocol and demanding pulse sequences standardization across different MR systems have prevented the reported method from being favorably accepted by the clinical community. Hence, an efficient method is proposed here to derive R^{a}₂ (θ) based on a single T2W sagittal image, by eliminating an assumed constant isotropic R₂ contribution derived from the magic angle locations in the deep cartilage. The new method is a unique Anisotropic R₂ of CollAgen DEgeneration (ARCADE) mapping to emphasize its straightforward association with the integrity of collagen fibers. The derived femoral cartilage R^{a}₂ (θ) values in five knees from four volunteers were compared with those of R₂−R_{1ρ}. The results demonstrate that the proposed ARCADE mapping is an efficient alternative to the conventional approach, holding great promise in providing both high-resolution morphological and more sensitive R^{a}₂ (») imaging from a single T2W scan in clinical studies on joint cartilage.

Theory

For knee cartilage water proton MR relaxation study at 3T, only the intramolecular dipolar interaction between two protons in water will be considered to interpret the observed MR relaxation rates of R₁, R₂, and R1_{ρ}. In general, these relaxation rates could be characterized using a two-pool fast exchange model, i.e., rapid water exchange between the “free” and “bound” pools, and thus represented as the weighted averages of two pools, as shown in Equation 1,

Rₘ=fi*Rⁱₘ+fb*R^{a}ₘ (θ) (1)

with m=1, 2 and 1_{ρ}; fᵢ and f_{b} the molecular fractions of water in the “free” and “bound” pools, with fi+fb=1; Rⁱₘ and R^{a}ₘ (θ) the contributions from a fast isotropic and a slow anisotropic molecular reorientation, which could be characterized, respectively, by a smaller effective isotropic correlation time (r_{f}) and a larger apparent isotropic correlation time (τ_{b}»τ_{f}). To simplify the discussion, Rⁱₘ and R^{a}ₘ (θ) can absorb corresponding fᵢ and f_{b}, to denote the “apparent” relaxation rates in the following unless stated otherwise.

The molecular anisotropic reorientation of the “bound” water in cartilage can be characterized using an axially symmetric model, with a correlation time τ_{∥} assigned to one rotation about the symmetry axis along the collagen fiber, and another correlation time τ_{⊥} to the rotation about an axis perpendicular to the symmetry axis. If τ_{∥} is set to τ_{f} and much smaller than τ_{⊥} (i.e. τ_{⊥}»τ_{∥})), corresponding to the “bound” water preferential alignments, the effective correlation time τ_{b} of the “bound” water could be determined only by τ_{⊥} leading to τ_{b}»τ_{f}. This conclusion had been long before stated that the preferential alignments of water molecules could effectively have their otherwise-short correlation times amplified by many orders of magnitude. Therefore, it would not be surprising to see a significantly larger τ_{b} for the restricted water in the “bound” pool.

R^{a}₂ (θ) can be explicitly written as R^{a}₂*(3 cos² θ−1)²/4, where R^{a}₂ denotes the maximum anisotropic relaxation rate and θ an angle subtending the dipolar interaction vector and the B₀ direction. R^{a}₂ (θ) reportedly could be effectively suppressed in R_{1ρ} measurements if using a stronger (ω₁/2π>2.0 kHz) spin-lock RF strength. Accordingly, an effective isotropic correlation time τ_{b} for the “bound” water could be estimated to be at least larger than 0.5/ω₁. On the other hand, the corresponding τ_{f} for the “free” water should be at least larger than 0.62/ω₀, given that R₂»R₁. The Larmor frequency is denoted by ω₀/2π and equal to 128 MHz at 3T.

According to the classical MR relaxation theories, water proton relaxation rates of R₁, R₂, and R_{1ρ} could be expressed in terms of an effective isotropic rotational correlation time τ_{c} using Equations 2-4, and profiled correspondingly at 3T,

$$\begin{matrix}{R}_{1} = K \{ \frac{{τ}_{c}}{1 + {ω}_{0}^{2} {τ}_{c}^{2}} + \frac{4 {τ}_{c}}{1 + 4 {ω}_{0}^{2} {τ}_{c}^{2}} \} & ( 2 ) \\ {R}_{2} = K \{ \frac{3 {τ}_{c}}{2} + \frac{2.5 {τ}_{c}}{1 + {ω}_{0}^{2} {τ}_{c}^{2}} + \frac{{τ}_{c}}{1 + 4 {ω}_{0}^{2} {τ}_{c}^{2}} \} & ( 3 ) \\ {R}_{1 ρ} = K \{ \frac{1.5 {τ}_{c}}{1 + 4 {ω}_{1}^{2} {τ}_{c}^{2}} + \frac{2.5 {τ}_{c}}{1 + {ω}_{0}^{2} {τ}_{c}^{2}} + \frac{{τ}_{c}}{1 + 4 {ω}_{0}^{2} {τ}_{c}^{2}} \} & ( 4 )\end{matrix}$$

where K is a constant of 1.056*10¹⁰ (s⁻²) assuming a distance of 1.59 (Å) between two proton nuclei in water. If τ_{c}«0.62/ω₀ (˜0.8*10⁻⁹ s), all relaxation rates will become 5Kτ_{c}, which describes a scenario for water molecules rotating freely in nonviscous liquids. In cartilage, however, water can attain a longer τ_{c}, depending on both interactions with its neighboring macromolecules and their relative orientations to B₀.

For the “free” water (0.62/ω₀<τ_{f}<0.5/ω₁) in cartilage, R^{j}_{1ρ} is equal to R^{j}₂, while R^{j}₁ becomes progressively smaller than R^{j}₂ as τ_{f} increases. Notably, R^{a}₁ for the “bound” water becomes insignificant, implying that the R₁ relaxation metric would be orientation-independent and only sensitive to the “free” water. For the “bound” water (τ_{b}>0.5/ω₁) in cartilage, R^{a}_{1ρ}(θ) is progressively decreased relative to R^{a}₂ (θ) as τ_{b} grows. In this case, R^{a}_{1ρ} (θ) can be recast by R^{a}₂ (θ)/1+4 ω₁²τ_{c}²) because of the dominant first term on the right side of Equations 3-4. It is worth mentioning that R_{1ρ} will turn into R₂ (i.e. R^{j}₂+R^{a}₂ (θ)) and R^{j}₂, respectively, when a spin-lock RF is absent and a stronger (ω₁²τ_{c}²»0.5) spin-lock RF is present. Consequently, the reported composite relaxation metric R₂−R_{1ρ} can be expressed in terms of R^{a}₂ (θ) as shown in Equation 5, predicting that R₂−R_{1ρ} would be a partial R^{a}₂ (θ) if a spin-lock RF strength is limited in clinical R_{1ρ} mapping.

R₂−R_{1ρ}=R₂^{a}(θ)*{4ω₁²τ_{c}²/(1+4ω₁²τ_{c}²)} (5)

The present disclosure provides an efficient alternative to derive R^{a}₂ (θ) from one T2W sagittal image, assuming constant proton density (S₀) and R^{j}₂ in cartilage. Typically, an orientation-dependent signal intensity S(θ) in T2W could be written as shown in Equation 6, including both “free” and “bound” water contributions, with TE being an echo-time.

S(θ)=S_{θ} exp(−TE*R₂ⁱ−TE*R₂^{a}(θ)) (6)

S(θ=±54.7°)=S_{θ} exp(−TE*R₂¹) (7)

R₂^{a}(θ)={log(S(θ=±54.7°))−log(S(θ))}/TE (8)

As collagen fibers in the DZ are oriented predominately perpendicular to the cartilage surface, R^{a}₂ (θ) will become zero at the magic angles of ±54.7. In this case, Equation 6 will reduce to Equation 7, which represents the “free” water contribution as an internal reference of the assumed constant S₀ exp (−TE*R^{j}₂) in the deep cartilage. Combining Equation 6 and Equation 7, R^{a}₂ (θ) can be computed using Equation 8. This proposed method has leveraged the specific femoral cartilage geometric information that can substitute the otherwise-required additional T2W measurement in conventional R₂ mapping.

The preliminary comparative results from five knees in a study described below demonstrate that a higher quality relaxation metric map with an enhanced contrast and less motion artifacts could be obtained with a minimum cost to clinical MR scan time. The proposed method could be potentially applied to other human joint cartilage studies such as in hips and elbows.

### Materials and Methods

### Volunteer Subjects

Four volunteers (S1-S4) were enrolled in a study in which each volunteer had a single knee scanned (except for S4, who had both knees studied). Five datasets were labeled according to the subject's sex (M/F), age, knee (L/R), and health status (symptomatic [S], asymptomatic [A] or ACL repaired [P]), i.e. S1 M52RA, S2M47LS, S3F41 LA, S4F20LP and S4F20RA. The study was approved by the local institutional review board (IRB) and compliant with the Health Insurance Portability and Accountability Act (HIPAA). Each volunteer was informed about the study and signed a consent form.

### MR Imaging Protocols

Both R₂ and R_{1ρ} mappings were performed using a dedicated 16 Channel T/R Knee Coil on a 3T MR scanner. 3D images with various spin-echo times (TEs) in R₂ mapping and spin-lock durations (TSLs) in R_{1ρ} mapping were collected in sagittal planes. A fast parallel imaging with an acceleration factor of 2 was used in both data acquisitions.

R₂ Mapping:

An interleaved multi-slice (=43) multi-echo (=8) turbo spin-echo (TSE) pulse sequence was used to collect 3D T2W images (FOV=128*128*128 mm³) with a voxel size of 0.6*0.6*3.0 mm³ covering entire tibiofemoral compartments. The reconstructed images were interpolated to a final voxel resolution of 0.24*0.24*3.00 mm³. An effective TE for volumetric image data of each was n*6.1 ms, with n=1, 2, 3, 4, 5, 6, 7, 8. The pulse repetition time was 2500 ms, and a total scan time was about 9 minutes per knee.

R_{1ρ} Mapping:

A spin-lock prepared T₁-enhanced 3D turbo gradient-echo sequence (T₁-TFE) was used to acquire T1ρ-weighed images through a segmented elliptic-centric k-space acquisition. The specific k-space trajectories moved along radial directions in a zigzag pattern starting from the center to optimally sample the spin-lock prepared signals before a steady-state was established. The spin-lock RF field strength (ω₁=γB₁/2π) was 500 Hz, and TSL were 0, 10, 20, 30 and 40 ms, respectively. A field of view was 130*130*130 mm³ (an acquired voxel size of 0.40*0.40*3.00 mm³ and interpolated to 0.24*0.24*3.00 mm³). The total scan duration was about 11 minutes per knee.

### MR Image Post-Processing

### Image Co-Registrations:

A free software Elastix was used for intraseries and interseries image coregistrations. The T2W or T1ρ 3D images with different TE and TSL were first aligned within time series; then, the aligned T1ρ 3D images were further coregistered to the aligned T2W 3D images. The coregistration scheme was based on a published protocol for human knee cartilage, including a multiresolution approach and a rigid transformation model. The coregistration was optimized over 1,000 iterations using a localized mutual information (MI) as a similarity metric, and mutual information was progressively maximized by minimizing its negative values in the optimization process.

### Angular-Radial Segmentation:

First, a whole femoral cartilage was manually delineated using a free software ITK-SNAP for each slice in T2W and T1ρ 3D data. Region of interest (ROI) vertices were placed along cartilage boundaries, with the vertex path defined as smoothly as possible. Furthermore, nonoverlaid cartilage areas (due to motion or misalignment) in both T2W and T1ρ images were minimized in delineating cartilage ROI.

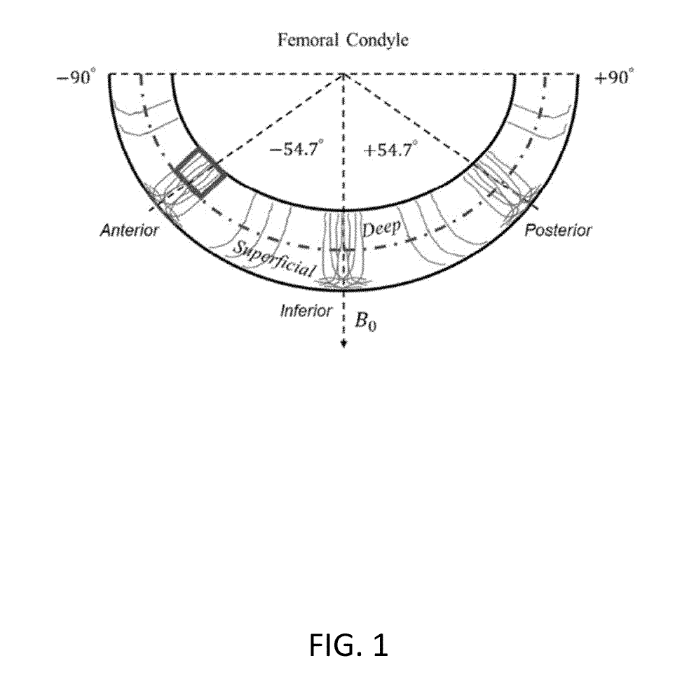

Second, the localized cartilage partitions were accomplished by an angular and radial segmentation method. Specifically, the vertices (x and y coordinates) from a predefined cartilage ROI were used to fit (by a nonlinear least-squares fitting) a virtual circle in each image slice, with the circle center located in the femoral condyle. Relative to a vertical line, an angle φ of a “spoke” connecting each vertex and the virtual center could be calculated; subsequently, the whole cartilage was subdivided angularly into 5° partitions based on the range of calculated “spoke” angles. A reference angle (0°) was chosen as the B₀ direction in a sagittal image, with the negative angles pointing to the anterior direction and the positive angles to a posterior direction (see FIG. 1).

Third, as the shape of femoral cartilage deviates from an ideal half-circle, especially on the lateral sides, a segmented angle (φ) had to be recomputed using only adjacent (φ=±10°) vertices to generate a new angle (θ) to represent collagen fiber orientation closely in the deep cartilage.

Finally, the femoral cartilage was further subdivided radially into two zones, i.e. the deep zone (DZ) and the superficial zone (SZ), with a shared border line equidistant to opposite boundaries. This segmented SZ actually covers at least both the histologically defined SZ and the transition zone (TZ).

R₂−R₁ Parametric Map:

Both R₂ and R_{1ρ} maps were fitted pixel by pixel from coregistered multiple 3D data using an exponential relaxation decay model, i.e., S(tᵢ)=S₀ exp(−tᵢ*P), where P=R₂ or R_{1ρ}, and tᵢ=[6.1, 12.2, 18.3, 24.4, 30.5, 36.6, 42.7, 48.8] or [0, 10, 20, 30, 40] (ms), respectively. The corresponding parametric error maps were also created by adjusting fitted parameter uncertainties so that the reduced χ² was equal to 1. A composite metric R₂−R_{1ρ} 3D parametric map was merely a difference between the fitted R₂ and R_{1ρ} 3D maps. All data analysis and image visualization were performed using an in-house software developed in IDL 8.5 (Harris Geospatial Solutions, Inc., Broomfield, Colo.).

R(θ)₂^{a} Parametric Map from ARCADE:

A single T2W (TE=48.8 ms) 3D dataset from R₂ mapping was used. An internal reference corresponding to the “free” water contribution for each image slice was estimated using Equation 6. Specifically, the average T2W pixel values (in logarithmic scale) within each of segmented ROIs in the DZ were fitted to a function of collagen fiber orientations (θ) as shown in Equation 9, with parametric bound constraints.

y=A−B*(3 cos²(θ+C)−1)² (9)

The model parameter A was not constrained; however, B and C were limited to the ranges of [0, 10] and [−10°, +10°], respectively. The limited freedom introduced for θ was to account for potential systematic errors in collagen fiber orientations.

The nonlinear curve fitting was performed slice by slice. The optimal fits were determined using goodness of fits characterized by χ² test statistics with a significant level of P>0.95. Finally, the mean of those determined optimal A values was used as a global internal reference, i.e., log (S(θ=54.7°) in Equation (8).

### Statistical Analysis:

The differences and associations between two relaxation metrics were, respectively, quantified using a Student paired t test (a two-tailed distribution) and a Pearson correlation coefficient (r), where the statistical significance was considered at P<0.05. Scatterplots were used to demonstrate the potential correlation between two parameters; additionally, data ellipses with a 95% confidence level were included for visual enhancement. A normalized relaxation metric, in terms of zonal contrast, was generated as (DEEP−SURF)/DEEP*100%, with DEEP and SURF representing R(θ)₂^{a} or R₂−R_{1ρ} in the DZ and SZ, respectively. All measurements are shown as mean±SD unless stated otherwise.

### Results:

A schematic sketch in FIG. 1 illustrates orientations of collagen fibers in the deep and the superficial zones, anatomical locations and angular-radial segmentations on a perfectly circular femoral cartilage in a sagittal plane. A red square at a magic-angle in the deep cartilage is highlighted where resides an internal reference signal.

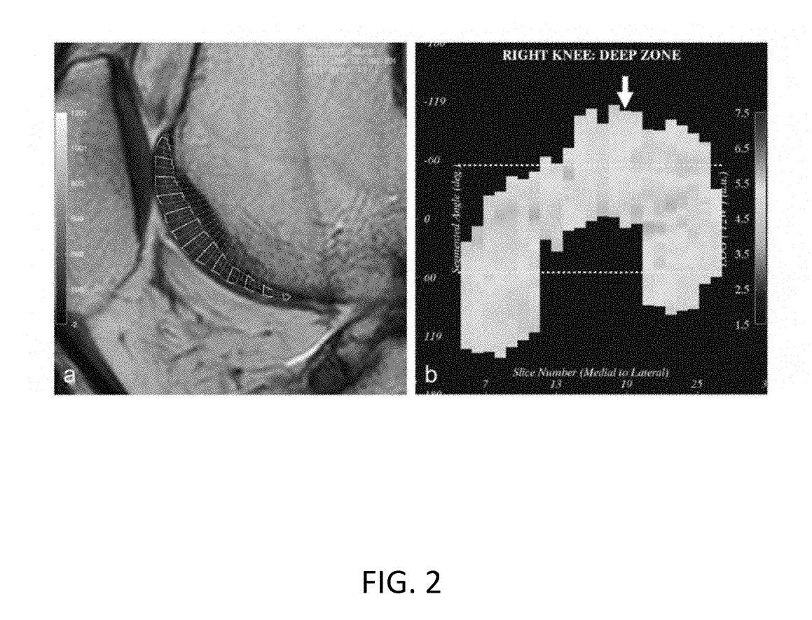

A segmentation example of a lateral image from one subject's right knee (S1M52RA) is shown in FIG. 2a, which overlays its corresponding T2W image. An exact spatial location of this image is specified by a white arrow on a whole deep cartilage T2W intensity (in a logarithmic scale) map as shown in FIG. 2b. The two horizontal dashed lines represent segmented angles φ of ±54.7°. The internal reference signal for this knee was estimated to be 5.52.

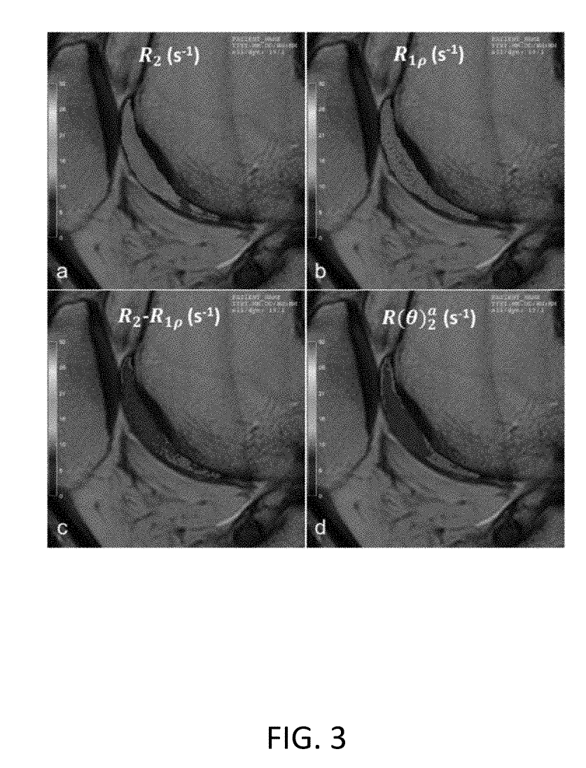

FIG. 3d shows R(θ)₂^{a} pixel map derived using Eq. 3 for the image in FIG. 2a, and was compared to a composite relaxation metric of R₂−R_{1ρ} in FIG. 3c. Clearly, R(θ)₂^{a} qualitatively reproduced its counterpart of R₂−R_{1ρ} with less motion artifacts, thanks to a significant fast (i.e. 1.2 vs. 20 minutes) acquisition of one T2W 3D image data. As a reference, original R₂ and R_{1ρ} pixel maps are listed respectively in FIG. 3a and FIG. 3b, which primarily resonated with previous findings in that the relaxation anisotropy was largely suppressed in R_{1ρ}.

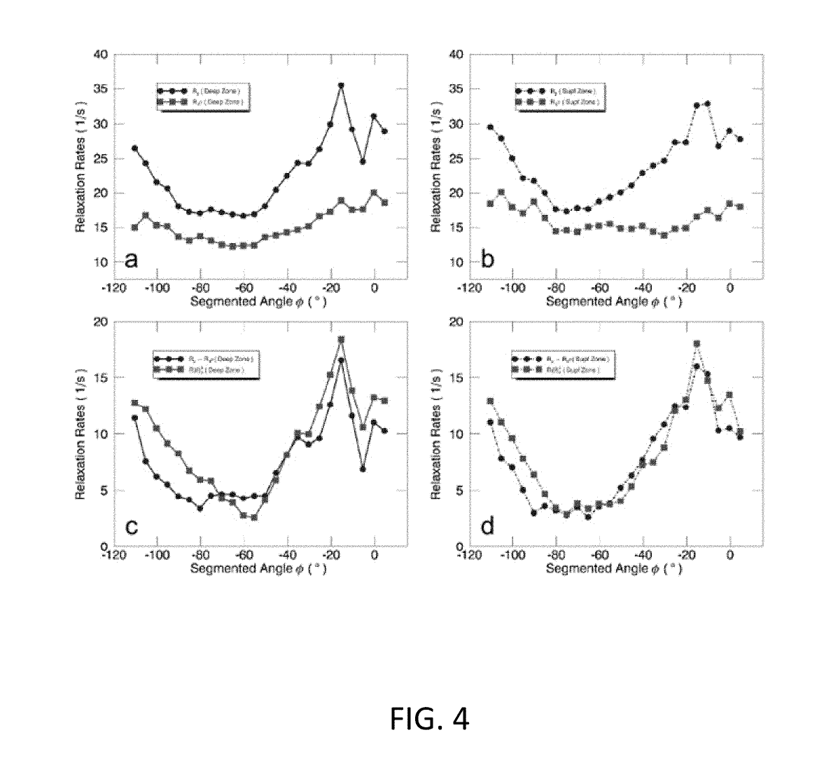

The relaxation anisotropy profiles are plotted in FIG. 4, which were obtained by averaging ROI values in the angular-radial segmentations in FIG. 3. As expected, R(θ)₂^{a} values were significantly higher than those of R₂−R_{1ρ} in both the deep (9.17±4.26 vs. 7.58±3.43, ρ<0.001, see FIG. 4c) and the superficial (8.35±4.36 vs. 7.63±4.12, p=0.04, se FIG. 4d) zones. On the other hand, R_{1ρ}, relative to R₂, had been significantly suppressed in both the deep (22.72±5.43 vs. 15.14±2.28, p<0.001, see FIG. 4a) and the superficial (23.77±4.79 vs. 16.14±1.75, p<0.001, see FIG. 4b) zones.

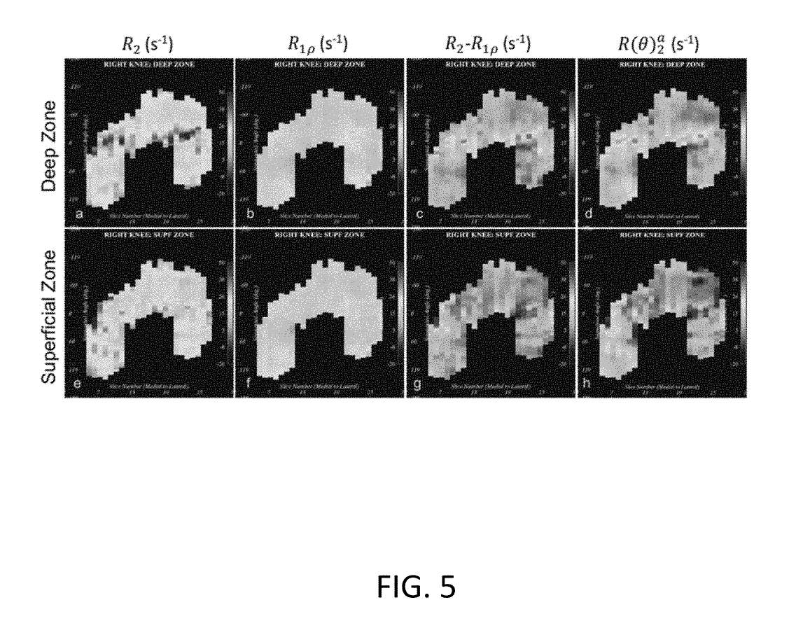

Moving forward, the complete whole knee maps of R₂ (FIG. 5a,e), R_{1ρ} (FIG. 5b,f), R₂−R_{1ρ} (FIG. 5c,g) and R(θ)₂^{a} (FIG. 5d,h) in the deep (FIG. 5a-d) and the superficial (FIG. 5e-h) zones are presented in FIG. 5. On a global scale, R(θ)₂^{a} qualitatively resembled R₂−R_{1ρ} in both cartilage zones and all four relaxation rates were relatively higher in the deep zone (FIG. 5a-d vs. FIG. 5e-h). It is worth noting that R_{1ρ} maps, on the same color scales, were much more featureless compared with R₂ maps.

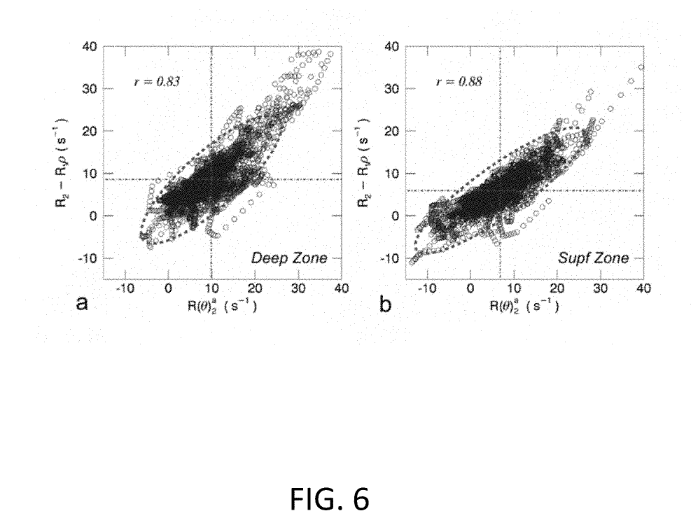

Quantitatively, R(θ)₂^{a} in this exampled cartilage highly correlated with R₂−R_{1ρ}, as shown in FIG. 6, in both the deep (r=0.83 in FIG. 6a) and the superficial (r=0.88 in FIG. 6b) zones. The averaged R(θ)₂^{a} values were slightly higher than those of R₂−R_{1ρ} in the deep (9.92 vs. 8.57 s⁻¹) and the superficial (6.84 vs. 5.95 s⁻¹) zones as specified by the data ellipse centroids.

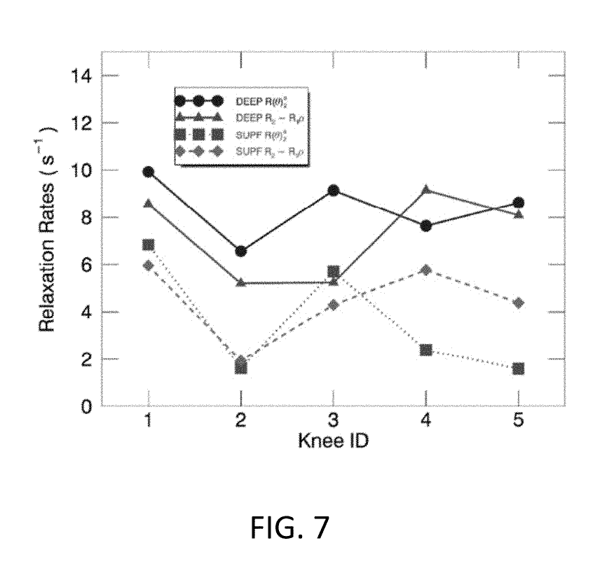

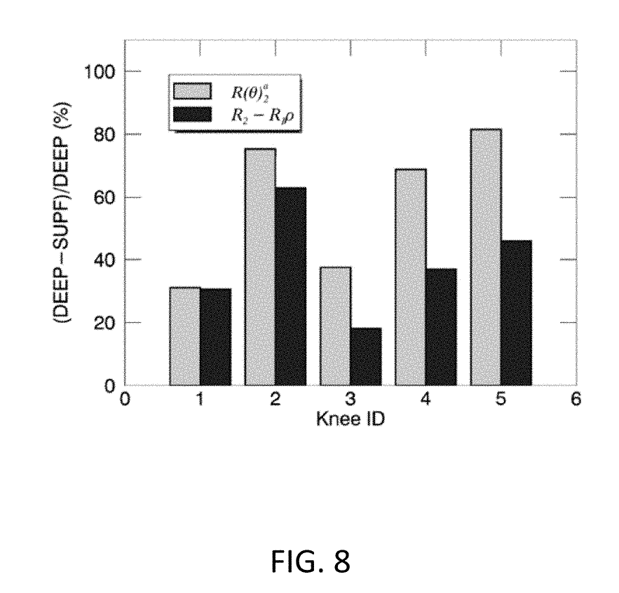

The relationship between an averaged R(θ)₂^{a} and an averaged R₂−R_{1ρ} for five knees are presented in FIG. 7, indicating a highly positive correlation (r=0.83±0.07) in both subdivided cartilage zones. Although R(θ)₂^{a} was slightly but not significantly higher than or hardly differentiated from R₂−R_{1ρ} in the deep (8.38±1.31 vs. 7.25±1.89, p=0.26) and the superficial (3.63±2.47 vs. 4.46±1.61, p=0.44) zones, the corresponding averaged relaxation metrics normalized between the two subdivided zones, defined as (DEEP−SUPF)/DEEP*100% as shown in FIG. 8, was significantly higher for R(θ)₂^{a} relative to R₂−R_{1ρ} (i.e. 59±23% vs. 39±17%, p<0.04), implying an enhanced image contrast in the derived relaxation metric using the developed method.

In summary, the comparative relaxation results on five femoral cartilages have demonstrated that R(θ)₂^{a} derived from the proposed method could be an efficient alternative to R₂−R_{1ρ}, which could provide a higher quality diagnostic-relevant relaxation parametric map at a minimum cost to routine clinical MR scan time.

### Discussion:

In the present disclosure, it was established that the composite relaxation metric (R₂−R_{1ρ}) actually measures inefficiently a partial anisotropic R₂ (R(θ)₂^{a}) in clinical knee cartilage studies at 3T. Accordingly, the present disclosure introduces a new method to extract an un-compromised R(θ)₂^{a} based on a single T2W sagittal image. The comparable results between the derived R(θ)₂^{a} and the measured R₂−R_{1ρ} on five femoral cartilages demonstrate that the developed method can be an efficient alternative to the conventional approach.

A key assumption in the new method was a uniform proton density S₀ and a constant isotropic R₂ (R₂ⁱ) regardless of its locations and health status in articular cartilage, where the differences in observed R₂ relaxation rates stemmed solely from the “bound” water on differently orientated collagen fibers. This oversimplified view on the “free” water was mainly based on previous observations in that the estimated water content and the observed R₂ values at the magic angle (MA) orientations were all nearly uniform across different zones in cartilage.

For example, one ex vivo study showed that S₀ was marginally larger in the SZ than in the DZ (i.e. 90±3% vs. 88±4%) at a location near the MA. Xia reported an approximately constant T₂ (59±6 ms) in cartilage specimens when orientated at the MA in a high-resolution μMRI study. Xia also found that T₁ was orientation-independent and almost constant (1.72±0.11 sec), as was confirmed recently by Hänninen et al. Based on MR relaxation theories, both isotropic R^{j}₂ and R^{j}₁ have nearly linear relationships albeit opposite with an effective correlation time τ_{f} of the “free” water in tissue. Thus, a uniform R₁ could be reasonably interpreted as a constant R^{j}₂, as the observed R₁ in cartilage is predominantly contributed from R^{j}₁.

As articular cartilage has a similar biochemical composition and structural network in extracellular matrix, the “free” water contribution to R₂ should not substantially fluctuate in different cartilages; in other words, the internal reference derived from the deep femoral cartilage is applicable to the tibial and patellar cartilages as well. The preliminary data (not shown) indicated that comparable correlations between R(θ)₂^{a} and R₂−R_{1ρ} were found in the femoral, tibial, and patellar cartilages, and an average R(θ)₂^{a} in the tibial was almost three times larger than those found in the femoral and patellar cartilages.

The assumption used in ARCADE is no exception for an OA population. The integrity of collagen fibers could be compromised as a result of pathology leading to less preferentially orientated water, and the amount of released “free” water would be very small compared to an existing large pool of free water. Although the free water contribution to T2W signal should not be altered in OA subjects, the observed T₂ at the locations other than the MA orientations could be increased and could adversely impact optimal curve fittings in some image slices and thus potentially lead to a biased internal reference. On the other hand, if localized OA happens to be at the MA sites, the internal reference would not be altered unless an insignificant (<4% at 3T) chemical exchange effect associated with GAG loss were taken into account.

Had the assumption been violated, the derived R(θ)₂^{a} would have been offset systematically from its true value as the R(θ)₂^{a} computation was just a simple subtraction in logarithmic scale. In this work, the measured R₂−R_{1ρ} was expected to be smaller than R(θ)₂^{a} because of a limited spin-lock RF strength used in R_{1ρ} mapping. According to previous reports, a spin-lock RF strength of 2.0 kHz could adequately (˜99%) suppress R(θ)₂^{a}, leading to R₂−R_{1ρ}=99%*R(θ)₂^{a}. In clinical R_{1ρ}studies at 3T, however, the spin-lock RF strength is usually limited to 0.5 kHz, which would translate into R₂−R_{1ρ}≈86%*R(θ)₂^{a}. On the other hand, the observed R₂−R_{1ρ} could be erroneously increased due to different data acquisitions, where R₂ derived from a multiecho pulse sequence tends to be more overestimated than R_{1ρ} from a pulse sequence similar to 3D-MAPSS. Consequently, the enhanced R₂−R_{1ρ} could compensate for the loss due to a limited spin-lock RF power, which might justify the comparable R(θ)₂^{a} and R₂−R_{1ρ} observed for some subjects in the current study.

The derived R(θ)₂^{a} values generally agreed with the prediction in the DZ except for the first knee (S1). It was quite likely that the determined internal references for S1 in the DZ and for others in the SZ were underestimated, leading to an unexpected smaller R(θ)₂^{a}. However, when using a normalized relaxation metric in terms of zonal contrast, all systematic errors associated with the internal reference and pulse sequences became irrelevant, and the derived normalized R(θ)₂^{a} from ARCADE was significantly larger than the normalized R₂−R_{1ρ} as predicted. Furthermore, the observed variations in both R(θ)₂^{a} and R₂−R_{1ρ} might reflect collagen fiber unique arrangements due to the volunteers' different ages (20-52 years old), sexes (M/F), and knee health statuses (ACL repaired, asymptomatic, or symptomatic). Even though their true relaxation rates might be systematically offset, the reported significant correlations should not be impaired.

R(θ)₂^{a} was found to be qualitatively comparable to R₂−R_{1ρ} as shown in FIG. 3 and FIG. 5; quantitatively, both were highly correlated as demonstrated in FIG. 6 and FIG. 7, where a relatively larger R(θ)₂^{a} was detected for the majority of five deep cartilage as illustrated in FIG. 7. Additionally, the proposed method was shown providing a higher contrast R(θ)₂^{a} map with less motion artifacts. Accordingly, a significant time saving in clinical MR scans and much less efforts in the image post-processing adds a strong impetus for further validating the new method in a lager clinical study.

Currently, it usually takes about 20 minutes to complete both R₂ and R_{1ρ} mapping acquisitions for just one knee. During this lengthy scan duration, patients face a notable challenge of keeping their knees still. Consequently, the motion artifacts induced during data acquisitions and later image post-processing (i.e. co-registrations) were inevitable as shown in FIG. 3c. In contrast, the proposed method depends only on one T2W 3D image acquisition that cost just 1.2 minutes in the study, thus, the potential motion artifacts were significantly reduced as demonstrated in FIG. 3d. With the proposed method, an isotropic high-resolution R(θ)₂^{a} map could be obtained, which could further improve sensitivities of the compositional MR imaging to cartilage degenerative changes.

In articular cartilage, collagen fibers are orientated predominantly perpendicular to and parallel to cartilage surface in the deep and the superficial zones, respectively. On the contrary, collagen fibers have no preferential orientations in the transitional zone. In our subdivided superficial zone, the majority of collagen fibers was essentially located in the histologically defined transitional zone, thus, the magic angle effect was relatively small. This was the very reason why a comparatively similar anisotropic R₂ values in the superficial zone in FIG. 7, and an amplified contrast within cartilage in FIG. 8 were observed.

As demonstrated in FIG. 7, the majority (4 out 5) of whole knee averaged R(θ)₂^{a} was larger than those obtained using the conventional approach in the deep cartilage. It is worth noting that the subjects 2 and 4 (i.e. S2M47LS and S4F20LP) had either knee pain or ACL repair surgery history, whose R(θ)₂^{a} values happened to be relatively smaller in the deep cartilage. According to a recent study, R(θ)₂^{a} seems to have a best sensitivity in detecting tissue changes related to OA. To draw a meaningful conclusion, a larger sample size in measured R(θ)₂^{a} would be needed in the future investigations.

R₂ and R_{1ρ} mappings are the most widespread pre-structural and compositional MR techniques in evaluating knee early cartilage degeneration in an expanding aging population and young athletes after sport injuries and surgical reconstruction. Although these advanced MR imaging techniques have been available for more than two decades, they are still not favorably accepted in the routine clinical practice due mainly to MR technical challenges. The proposed approach is basically an internal reference method; hence, all systemic errors reported in R₂ and R_{1ρ} mappings induced by various pulse sequence implementations on different platforms will vanish in the new method, making it a standardized R(θ)₂^{a} measurement much easier in a multi-center trial. Finally, this developed MR method to extract a meaningful relaxation metric could be applied equally to other human joint cartilage such as those in hips and elbows.

As an internal reference method, the developed ARCADE mapping should alleviate any systematic errors known for both R₂ and R_{1ρ} mappings due to different pulse sequence implementations on multiple platforms, making it easier to standardize R(θ)₂^{a} measurement in a multicenter trial and be integrated in clinical studies. This new method is independent of the pulse sequence implementation as long as the image pixel intensities are spin-echo weighted, which is inherently insensitive to B₀ inhomogeneity. Advanced knee coil provides an excellent B₁ homogeneity, with less than ˜5% variations in flip angle reported across the cartilage regions of interest in the sagittal plane. Therefore, ARCADE could be reasonably considered to be insensitive to both B₀ and B₁ inhomogeneities. In short, an isotropic high-resolution 3D morphological and more relevant and sensitive R(θ)₂^{a} relaxation metric imaging could be fore-seen with a single scan in a clinical setting for both knee and other joints

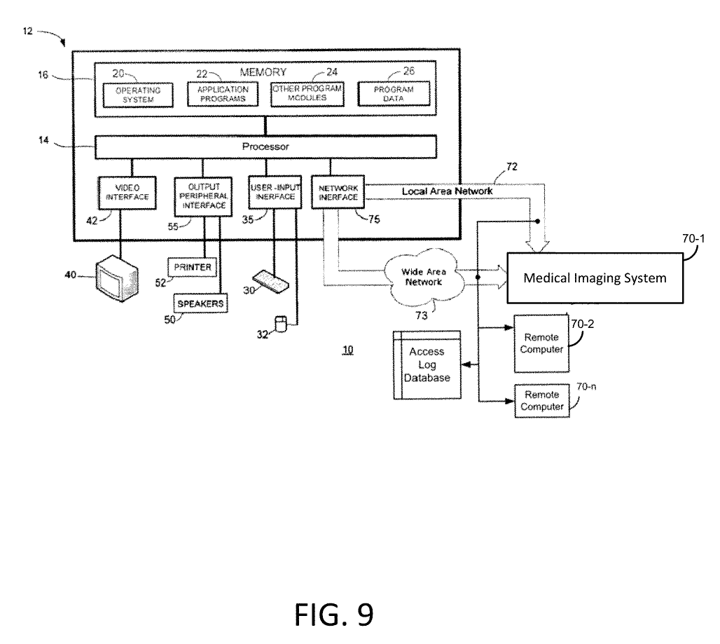

With reference to FIG. 9, an exemplary system for implementing the blocks of the method and apparatus includes a general-purpose computing device in the form of a computer 12. Components of computer 12 may include, but are not limited to, a processing unit 14 and a system memory 16. The computer 12 may operate in a networked environment using logical connections to one or more remote computers, such as remote computers 70-1, 70-2, . . . 70-n, via a local area network (LAN) 72 and/or a wide area network (WAN) 73 via a modem or other network interface 75. These remote computers 70 may include other computers like computer 12, but in some examples, these remote computers 70 include one or more of (i) a medical imaging system, such as magnetic resonance imaging (MRI) device, (ii) a signal records database systems, (iii) a scanner, and/or (v) a signal filtering system.

In the illustrated example, the computer 12 is connected to a medical imaging system 70-1. The medical imaging system 70-1 may be a stand-alone system capable of performing imaging of molecules, such as water, in biological tissue for in vivo examination. The system 70-1 may have resolution of such biological features as fibers, membranes, micromolecules, etc., wherein the image data can reveal microscopic details about biological tissue architecture, in a normal state or diseased state.

Computer 12 typically includes a variety of computer readable media that may be any available media that may be accessed by computer 12 and includes both volatile and nonvolatile media, removable and non-removable media. The system memory 16 includes computer storage media in the form of volatile and/or nonvolatile memory such as read only memory (ROM) and random access memory (RAM). The ROM may include a basic input/output system (BIOS). RAM typically contains data and/or program modules that include operating system 20, application programs 22, other program modules 24, and program data 26. The computer 12 may also include other removable/non-removable, volatile/nonvolatile computer storage media such as a hard disk drive, a magnetic disk drive that reads from or writes to a magnetic disk, and an optical disk drive that reads from or writes to an optical disk.

A user may enter commands and information into the computer 12 through input devices such as a keyboard 30 and pointing device 32, commonly referred to as a mouse, trackball or touch pad. Other input devices (not illustrated) may include a microphone, joystick, game pad, satellite dish, scanner, or the like. These and other input devices are often connected to the processing unit 14 through a user input interface 35 that is coupled to a system bus, but may be connected by other interface and bus structures, such as a parallel port, game port or a universal serial bus (USB). A monitor 40 or other type of display device may also be connected to the processor 14 via an interface, such as a video interface 42. In addition to the monitor, computers may also include other peripheral output devices such as speakers 50 and printer 52, which may be connected through an output peripheral interface 55.

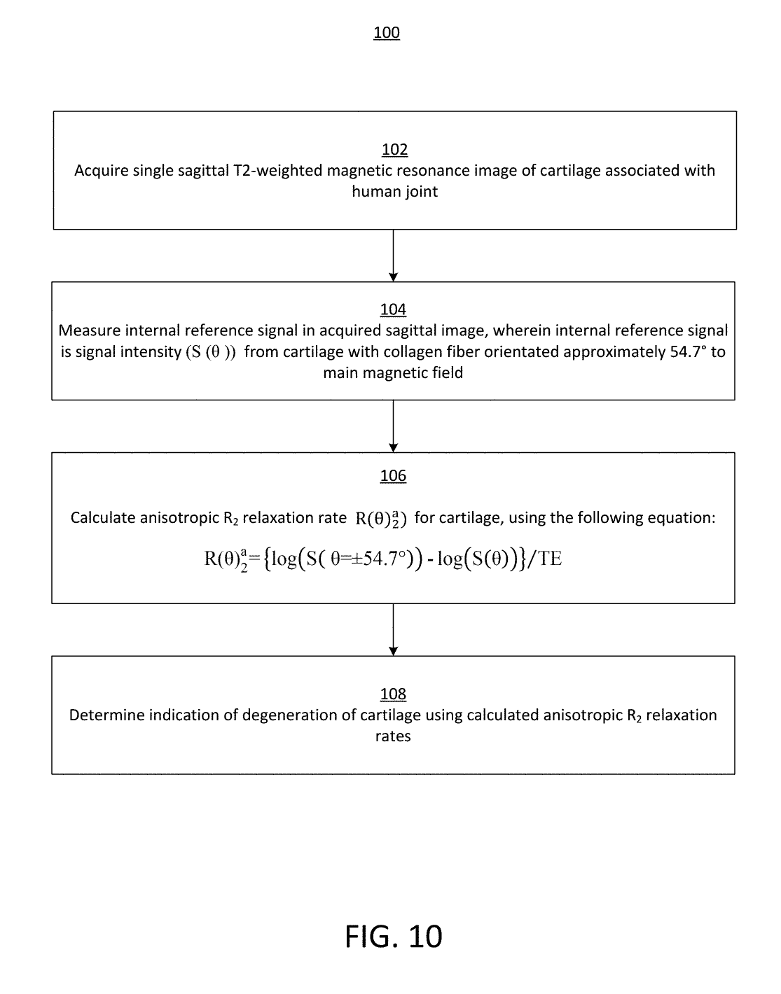

Referring now to FIG. 10, a flow diagram of an exemplary method for measuring clinically relevant Magnetic Resonance (MR) anisotropic water proton transverse relaxation rates of cartilage in human knee and other joints is illustrated, in accordance with an embodiment. A single sagittal T2-weighted magnetic resonance image of cartilage associated with a human joint may be acquired (block 102). For example, the cartilage may be associated with a human knee joint (e.g., femoral cartilage, tibial cartilage, patellar cartilage, etc.), a human hip joint, a human elbow joint, etc. An internal reference signal in the acquired sagittal image may be measured (block 104). Generally speaking, the internal reference signal is a signal intensity (S(θ)) from the deep femoral cartilage with its collagen fiber orientated approximately 54.7° to the main magnetic field. An anisotropic R₂ relaxation rate (R(θ)₂^{a}) may be calculated (block 106) for the human knee cartilage using the following equation:

R(θ)₂^{a}={log(S(θ=±54.7°))−log(S(θ))}/TE.

An indication of a degeneration of the cartilage may be determined (block 108) using the calculated anisotropic R₂ relaxation rates.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

Thus, although certain apparatus constructed in accordance with the teachings of the invention have been described herein, the scope of coverage of this patent is not limited thereto. On the contrary, this patent covers all embodiments of the teachings of the invention fairly falling within the scope of the appended claims either literally or under the doctrine of equivalents.

What is claimed is:

1. A computer-implemented method, comprising:
acquiring a single sagittal T2-weighted magnetic resonance image of cartilage associated with a human joint;
measuring an internal reference signal in the acquired sagittal image, wherein the internal reference signal is a signal intensity (S(θ)) from the cartilage with its collagen fiber orientated approximately 54.7° to the main magnetic field;
calculating an anisotropic R₂ relaxation rate (R(θ)₂^{a}) for the cartilage, using the following equation: R(θ)₂^{a}={log(S(θ=±54.7°))−log(S(θ))}/TE; and
determining an indication of a degeneration of the cartilage using the calculated anisotropic R₂ relaxation rates.

2. The method of claim 1, wherein the cartilage is associated with a human knee joint.

3. The method of claim 1, wherein the cartilage is associated with a human hip joint.

4. The method of claim 1, wherein the cartilage is associated with a human elbow joint.

5. The method of claim 2, wherein the cartilage is femoral cartilage.

6. The method of claim 2, wherein the cartilage is tibial cartilage.

7. The method of claim 2, wherein the cartilage is patellar cartilage.

8. A system, comprising:
one or more processors; and
one or more memories storing instructions that, when executed by the one or more processors, cause the one or more processors to:
acquire a single sagittal T2-weighted magnetic resonance image of cartilage associated with a human joint;
measure an internal reference signal in this acquired sagittal image, wherein the internal reference signal is a signal intensity (S(θ)) from the cartilage with its collagen fiber orientated approximately 54.7° to the main magnetic field;
calculate an anisotropic R₂ relaxation rate (R(θ)₂^{a}) for the cartilage, using the following equation: R(θ)₂^{a}={log(S(θ=±54.7°))−log(S(θ))}/TE; and
determine an indication of a degeneration of the cartilage using the calculated anisotropic R₂ relaxation rates.

9. The system of claim 8, wherein the cartilage is associated with a human knee joint.

10. The system of claim 8, wherein the cartilage is associated with a human hip joint.

11. The system of claim 8, wherein the cartilage is associated with a human elbow joint.

12. The system of claim 10, wherein the cartilage is femoral cartilage.

13. The system of claim 10, wherein the cartilage is tibial cartilage.

14. The system of claim 10, wherein the cartilage is patellar cartilage.

15. A tangible, non-transitory computer-readable medium storing executable instructions that when executed by at least one processor of a computing device, cause the computing device to:
acquire a single sagittal T2-weighted magnetic resonance image of cartilage associated with a human joint;
measure an internal reference signal in this acquired sagittal image, wherein the internal reference signal is a signal intensity (S(θ)) from the cartilage with its collagen fiber orientated approximately 54.7° to the main magnetic field;
calculate an anisotropic R₂ relaxation rate (R(θ)₂^{a}) for the cartilage, using the following equation: R(θ)₂^{a}={log(S(θ=±54.7°))−log(S(θ))}/TE; and
determine an indication of a degeneration of the a degeneration of the cartilage using the calculated anisotropic R₂ relaxation rates.

16. The tangible, non-transitory computer-readable medium of claim 15, wherein the cartilage is associated with a human knee joint.

17. The tangible, non-transitory computer-readable medium of claim 15, wherein the cartilage is associated with a human hip joint.

18. The tangible, non-transitory computer-readable medium of claim 15, wherein the cartilage is associated with a human elbow joint.

19. The tangible, non-transitory computer-readable medium of claim 16, wherein the cartilage is femoral cartilage.

20. The tangible, non-transitory computer-readable medium of claim 16, wherein the cartilage is tibial cartilage.
